(19) United States
(12) Patent Application Publication
(10) Pub. No.: US 2021/0353499 A1
(43) Pub. Date: Nov. 18, 2021

(54) ELECTROMAGNETIC PHYSIOTHERAPY APPARATUS OF CONCEPTION AND GOVERNOR VESSELS AND PHYSIOTHERAPY METHOD OF CONCEPTION AND GOVERNOR VESSELS

(71) Applicant: Dawei ZHANG, Yulin (CN)

(72) Inventor: Dawei ZHANG, Yulin (CN)

(21) Appl. No.: 17/143,167

(22) Filed: Jan. 7, 2021

(30) Foreign Application Priority Data
May 14, 2020 (CN) 202010408937.8

(51) Int. Cl. A61H 39/02

(52) U.S. Cl. CPC A61H 39/02; A61H 2201/1621; A61H 2201/165; A61H 2201/10; A61H 2039/005; A61H 2201/1611; A61H 2201/1638; A61H 2201/1642; A61H 2201/0103; A61H 2201/1607

(57) ABSTRACT

An electromagnetic physiotherapy apparatus of conception and governor vessels includes an electromagnetic generating device, and a torso model structure. The torso model structure is electrically connected to the electromagnetic generating device, and is configured to be worn by the user, and the torso model structure is provided with electromagnetic diaphragms at positions corresponding to acupuncture points and tissues of a human body. The torso model structure includes a head unit, a chest unit, a waist unit and a lower abdomen unit, and the head unit, the chest unit, the waist unit and the lower abdomen unit are connected through a main torso. A physiotherapy method of conception and governor vessels includes: enabling a bionic electromagnetism to synchronously act on a neuron area, a block and a chain to promote connections between synapses of the neuron area, the block and the chain.

### CROSS REFERENCE TO THE RELATED APPLICATIONS

This application is based upon and claims priority to Chinese Patent Application No. 202010408937.8, filed on May 14, 2020, the entire contents of which are incorporated herein by reference.

### TECHNICAL FIELD

The present invention belongs to the field of health care instruments, and more particularly, to an electromagnetic physiotherapy apparatus of conception and governor vessels. In addition, the present invention further relates to an electromagnetic physiotherapy method of conception and governor vessels.

### BACKGROUND

A physiotherapy apparatus, or physical therapy apparatus, is used to enable physical factors to act on a human body to produce beneficial effects for the human body. The common physical factors include electricity, sound, light, magnetism, water, pressure, and the like. Compared with drug therapy, physical therapy has advantages of safety and environmental protection. Therefore, at present, physiotherapy apparatus have been widely used by individuals and families alike.

In the process of implementing the present invention, the inventor found that prior physiotherapy apparatus have at least the following disadvantages:

Prior physiotherapy apparatus are equipped with electrode patches, and when in use, it is necessary to attach the electrode patches to corresponding acupuncture points. For general users, there is a certain difficulty in determining the acupuncture points and accurately attaching the electrode patches to the acupuncture points, thereby influencing the effect of the physiotherapy. Additionally, prior electrode patches cannot meet a point-to-point physiotherapy method, which has an unsatisfactory physiotherapy effect.

### SUMMARY

Based on the above problems in the background, an objective of the present invention is to provide an electromagnetic physiotherapy apparatus of conception and governor vessels, which employs a fixed model structure. In use, the fixed model structure is directly sleeved on the human body, and thus directly corresponds to acupuncture points and tissues of the human body, thereby solving the professional problem of prior electrode patch and overcoming the disadvantage of the unsatisfactory physiotherapy effect of the point-to-point physiotherapy method. Another objective of the present invention is to provide an electromagnetic physiotherapy method of conception and governor vessels.

In order to achieve the above objectives, according to an aspect of the present invention, the embodiments of the present invention provide the following technical solutions.

An electromagnetic physiotherapy apparatus of conception and governing vessels includes an electromagnetic generating device and a torso model structure. The torso model structure is electrically connected to the electromagnetic generating device, and is configured to be worn by a user. The torso model structure is provided with electromagnetic diaphragms at positions corresponding to acupuncture points and tissues of a human body.

In an embodiment, the torso model structure includes a head unit, a chest unit, a waist unit and a lower abdomen unit. The head unit, the chest unit, the waist unit and the lower abdomen unit are connected through a main torso.

Preferably, the head unit has a helmet-shaped structure, and the head unit is provided with electromagnetic diaphragms at positions respectively corresponding to a prefrontal lobe, a posterior frontal lobe, a parietal lobe, a temporal lobe, an occipital lobe, a cerebellum, epithalamus and hypothalamus, a pituitary, an epiphysis, a pons and a medulla oblongata of a brain of the human body. Each of the chest unit, the waist unit and the lower abdomen unit has an annular structure, and the annular structure is provided with at least one electromagnetic diaphragm.

More preferably, the head unit includes: an arc belt, wherein the arc belt corresponds to a top of a head of the human body; a first annular belt, wherein the first annular belt is connected to the arc belt and is arranged around the head of the human body in one circle; a second annular belt, wherein the second annular belt is arranged around a neck of the human body in one circle; a third annular belt, wherein a top of the third annular belt is connected to the arc belt, and a bottom of the third annular belt is connected to the second annular belt; and a vertical belt, wherein a top of the vertical belt is connected to the first annular belt, and a bottom of the vertical belt is connected to the second annular belt. Each of the arc belt, the first annular belt, the second annular belt, the third annular belt and the vertical belt is provided with a plurality of electromagnetic diaphragms.

In an embodiment, the arc belt is further connected to a first branch belt and a second branch belt, and the first branch belt and the second branch belt are both perpendicular to an extension direction of the arc belt. The vertical belt is further connected to a third branch belt, a fourth branch belt and a fifth branch belt, and the third branch belt, the fourth branch belt and the fifth branch belt are all perpendicular to an extension direction of the vertical belt. Two ends of the first branch belt, two ends of the second branch belt, two ends of the third branch belt, two ends of the fourth branch belt and two ends of the fifth branch belt are separately provided with an electromagnetic diaphragm.

In an embodiment, the torso model structure further includes an auxiliary lead string, and the auxiliary lead string is configured to be tied to four limbs.

In an embodiment, the torso model structure is an inflatable structure, and the torso model structure in an inflated state enables the electromagnetic diaphragms to be tightly contact with the human body. The torso model structure is communicated with an inflation device. The torso model structure is provided with a joint, and the inflation device is communicated with the joint.

In an embodiment, the electromagnetic generating device includes an electromagnetic output unit and a power source. The electromagnetic output unit is electrically connected to the torso model structure, and the power source is electrically connected to the electromagnetic output unit.

In an embodiment, the electromagnetic physiotherapy apparatus of the conception and governor vessels further includes a control unit. The control unit communicates with the electromagnetic generating device to input physiotherapy modes, time and strength signals to control the running of the electromagnetic generating device.

According to another aspect of the present invention, the embodiments of the present invention provide an electromagnetic physiotherapy method of conception and governor vessels, including: enabling a bionic electromagnetism to synchronously act on a neuron area, a block and a chain to promote connections between synapses of the neuron area, the block and the chain. The neuron area includes a prefrontal lobe, a posterior frontal lobe, a parietal lobe, a temporal lobe and an occipital lobe of a brain. The block includes a cerebellum, epithalamus and hypothalamus, a pituitary, an epiphysis, a pons and a medulla oblongata. The chain includes a spinal cord and a peripheral nervous system.

Compared with the prior art, the embodiments of the present invention at least have the following effects:

1. The electromagnetic physiotherapy apparatus of conception and governor vessels of the present invention includes the torso model structure. In use, the torso model structure is directly sleeved on the human body, and thus has a corresponding function of acting on the corresponding acupuncture points and tissues of the human body, thereby simplifying the technical difficulty of professionality of the electrode patch and overcoming the disadvantage of the unsatisfactory physiotherapy effect of the point-to-point physiotherapy method.

2. The torso model structure of the present invention includes the head unit, the chest unit, the waist unit and the lower abdomen unit. The torso model structure is capable of enabling the bionic electromagnetism to directly act on the prefrontal lobe, the posterior frontal lobe, the parietal lobe, the temporal lobe, the occipital lobe, the cerebellum, the epithalamus and hypothalamus, the pituitary, the epiphysis, the pons, the medulla oblongata, the spinal cord, the nervous system and others of the brain, so as to promote connections between synapses of the neuron area (the prefrontal lobe, the posterior frontal lobe, the parietal lobe, the temporal lobe and the occipital lobe of the brain), the block (the cerebellum, the epithalamus and hypothalamus, the pituitary, the epiphysis, the pons and the medulla oblongata) and the chain (the spinal cord and the peripheral nervous system). In this way, the biological activity and function of each system are enhanced, the blood flow change and metabolism of each tissue system are improved, and the physiological activity and strain function of each tissue cell are improved.

3. The torso model structure of the present invention further includes an auxiliary lead string, and when in use, the lead string is tied to wrist portions of four limbs of the human body, which takes into account traditional electrode patch auxiliary leads.

4. The torso model structure of the present invention is an inflatable structure, and is capable of enabling the electromagnetic diaphragms to be tightly contact with the surface of the human body by means of the inflation adjustment of the inflation device.

### BRIEF DESCRIPTION OF THE DRAWINGS

In order to explain technical solutions in the embodiments of the present invention more clearly, drawings that need to be used in the description of the embodiments are simply introduced below.

FIG. 1 is a schematic diagram of the structure of an electromagnetic physiotherapy apparatus of conception and governor vessels according to Embodiment 1 of the present invention;

FIG. 2 is a schematic diagram of the structure of a head unit according to Embodiment 1; and

FIG. 3 is a schematic diagram of the structure of an electromagnetic physiotherapy apparatus of conception and governor vessels according to Embodiment 2 of the present invention.

### DETAILED DESCRIPTION OF THE EMBODIMENTS

In order to make the objectives, technical solutions and advantages of embodiments of the present invention be clearer, the technical solutions in the embodiments of the present invention will be described clearly and completely below in conjunction with the drawings in the embodiments of the present invention. Obviously, the described embodiments are a part of embodiments of the present invention, rather than the entire embodiments. Based on the embodiments of the present invention, all the other embodiments obtained by those having ordinary skill in the art without creative effort shall fall within the scope of protection of the present invention.

In the description of the present invention, it should be noted that orientations or position relationships indicated by the terms “center”, “top”, “bottom”, “left”, “right”, “vertical”, “horizontal”, “inner”, “outer”, “front”, “rear”, etc. are based on orientations or position relationships shown in the drawings, which are only for the convenience of describing the present invention and simplifying the description, and do not indicate or imply that the indicated devices or elements must have specific orientations or be constructed and operated in specific orientations, and thus cannot be understood as a limitation to the present invention.

In order to overcome the shortcomings of the prior electrode-patch physiotherapy apparatus, the present invention provides an electromagnetic physiotherapy apparatus of conception and governor vessels, including: an electromagnetic generating device and a torso model structure. The torso model structure is electrically connected to the electromagnetic generating device, and is configured to be worn by the user. The torso model structure is provided with electromagnetic diaphragms at positions corresponding to the acupuncture points and tissues of the human body. In use, the torso model structure is directly sleeved on the human body, and thus has a corresponding function of acting on the corresponding acupuncture points and tissues of the human body, thereby simplifying the technical difficulty of professionality of the electrode patch and overcoming the disadvantage of the unsatisfactory physiotherapy effect of the point-to-point physiotherapy method.

Next, the technical solutions of the present invention will be described in detail through specific embodiments.

### Embodiment 1

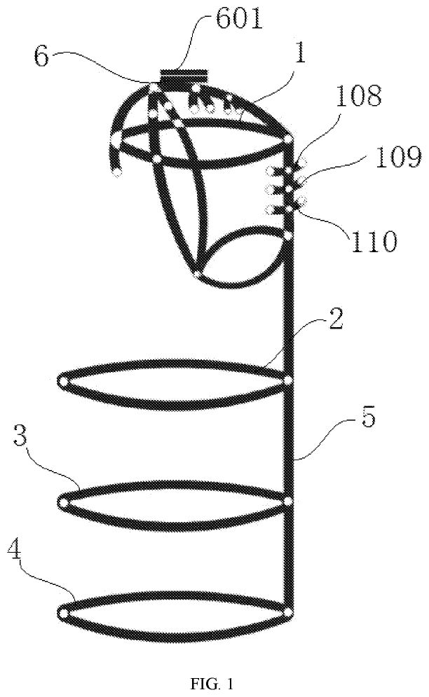

As shown in FIG. 1, an electromagnetic physiotherapy apparatus of conception and governor vessels includes an electromagnetic generating device and a torso model structure, and the torso model structure is electrically connected to the electromagnetic generating device.

In the present embodiment, the torso model structure includes the head unit 1, the chest unit 2, the waist unit 3 and the lower abdomen unit 4, and the head unit 1, the chest unit 2, the waist unit 3 and the lower abdomen unit 4 are connected through the main torso 5.

The torso model structure includes the head unit 1, the chest unit 2, the waist unit 3 and the lower abdomen unit 4. The head unit 1 is provided with electromagnetic diaphragms at positions respectively corresponding to the prefrontal lobe, the posterior frontal lobe, the parietal lobe, the temporal lobe, the occipital lobe, the cerebellum, the epithalamus and hypothalamus, the pituitary, the epiphysis, the pons and the medulla oblongata of the brain of the human body, so that the bionic electromagnetism output from the electromagnetic generating device is capable of acting on a cerebral cortex, a brain nucleus, a vital center, the cerebellum, the spinal cord and a nervous distribution system of the human body, and promoting the connections between the synapses of the neuron area (the prefrontal lobe, the posterior frontal lobe, the parietal lobe, the temporal lobe and the occipital lobe of the brain), the block (the cerebellum, the epithalamus and hypothalamus, the pituitary, the epiphysis, the pons and the medulla oblongata) and the chain (the spinal cord and the peripheral nervous system), thereby accurately improving the physiotherapy efficacy. The present invention has a characteristic of systematic function from a center of conception and governor vessels, has a substantial improvement in comparison with the traditional meridian physiotherapy apparatus, and also has a substantial distinction with the traditional electromagnetic physiotherapy apparatus using the heat effect.

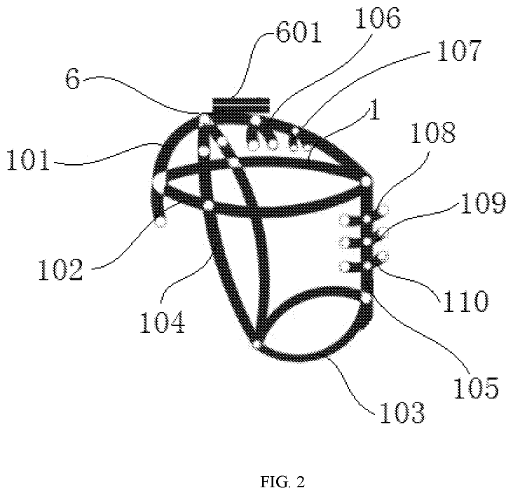

Specifically, as shown in FIG. 2, the head unit 1 has a helmet-shaped structure, and includes: the arc belt 101, the first annular belt 102, the second annular belt 103, the third annular belt 104 and the vertical belt 105.

The arc belt 101 corresponds to the top of the head of the human body, and has a radian matching with the top of the head of the human body. Six electromagnetic diaphragms (at the positions of the hollow circles in the FIG. 2) are distributed on the arc belt 101, wherein two electromagnetic diaphragms are disposed at two ends of the arc belt 101.

In order to improve the physiotherapy effect of the head, as shown in FIG. 2, the arc belt 101 is further provided with the first branch belt 106 and the second branch belt 107. The first branch belt 106 and the second branch belt 107 are perpendicular to an extension direction of the arc belt 101, and two ends of the first branch belt 106 and two ends of the second branch belt 107 are separately provided with an electromagnetic diaphragm.

The first annular belt 102 is connected to the arc belt 101. Taking FIG. 2 as an example, the left end of the first annular belt 102 is connected to the left side of the arc belt 101. The left end of the first annular belt 102 may be directly connected to the left end of the arc belt 101, or may be connected to the upper side of the left end of the arc belt 101, which are not specifically limited in the present embodiment. The right end of the first annular belt 102 is connected to the right end of the arc belt 101. That is, the first annular belt 102 in the present embodiment is arranged horizontally around the head of the human body in one circle. Four electromagnetic diaphragms are distributed on the first annular belt 102, wherein two electromagnetic diaphragms are located at two ends of the first annular belt 102, and the other two electromagnetic diaphragms correspond to positions of temples.

The left end of the second annular belt 103 is connected to the bottom of the third annular belt 104, and the right end of the second annular belt 103 is connected to the vertical belt 105. The second annular belt 103 is arranged around the neck of the human body in one circle, and two ends of the second annular belt 103 are separately provided with an electromagnetic diaphragm.

The top of the third annular belt 104 is connected to the arc belt 101, and the bottom of the third annular belt 104 is connected to the left end of the second annular belt 103. Three to four electromagnetic diaphragms are distributed on the third annular belt 104.

The top of the vertical belt 105 is connected to the right end of the first annular belt 102, and the bottom of the vertical belt 105 is connected to the right end of the second annular belt 103. The third branch belt 108, the fourth branch belt 109 and the fifth branch belt 110 are, from top to bottom, distributed on the vertical belt 105. The third branch belt 108, the fourth branch belt 109 and the fifth branch belt 110 are all disposed horizontally, that is, perpendicularly to the vertical belt 105. Two ends of the third branch belt 108, two ends of the fourth branch belt 109 and two ends of the fifth branch belt 110 are separately provided with an electromagnetic diaphragm. A joint of the vertical belt 105 and the third branch belt 108, a joint of the vertical belt 105 and the fourth branch belt 109 and a joint of the vertical belt 105 and the fifth branch belt 110 are separately provided with an electromagnetic diaphragm.

In the present embodiment, as shown in FIG. 1, each of the chest unit 2, the waist unit 3 and the lower abdomen unit 4 has an annular structure, and the right end of the chest unit 2, the right end of the waist unit 3 and the right end of the lower abdomen unit 4 are all connected to the main torso 5. Two ends of the chest unit 2, two ends of the waist unit 3 and two ends of the lower abdomen unit 4 are separately provided with an electromagnetic diaphragm. In order to match different body shapes and sizes of the users, the annular structure of each of the chest unit 2, the waist unit 3 and the lower abdomen unit 4 is adjustable.

It should be noted that the number and arranged positions of the electromagnetic diaphragms in the present embodiment are only described in a manner of taking examples, and specific use structures are not limited.

In the present embodiment, as shown in FIG. 1, the electromagnetic generating device includes the electromagnetic output unit 6 and the power source 601, and the electromagnetic output unit 6 and the power source 601 are both arranged at the top of the head unit 1. The power source 601 is electrically connected to the electromagnetic output unit 6 to supply power to the electromagnetic output unit 6, and the electromagnetic output unit 6 is electrically connected to the torso model structure to output electromagnetic signals.

The power source 601 may employ an external power wire. For the convenient use, the power source 601 may also be a storage battery or a dry battery, which are not specifically limited in the present embodiment. When the power source 601 is the storage battery, the power source 601 is electrically connected to a charging socket.

For the convenient running, the electromagnetic physiotherapy apparatus of conception and governor vessels in the present embodiment further includes a control unit, and the control unit communicates with the electromagnetic output unit 6. Specifically, the control unit in the present embodiment has a wristwatch structure, can be worn on the wrist, and is wirelessly connected to the electromagnetic output unit 6 to facilitate the user to select the physiotherapy modes, time, and strength signal to control the running of the electromagnetic output unit 6, wherein the physiotherapy mode may correspond to the treatment of different diseases.

It should be noted that the structure of the control unit is not limited to the above wireless wristwatch structure, and may also be a wired control panel structure in other embodiments. It should also be noted that the electromagnetic diaphragm, the electromagnetic output unit 6 and the wristwatch control unit in the present embodiment are all the prior products, and the specific structure and working principle thereof will not be described in detail in the present embodiment.

In use of the present embodiment, the electromagnetic diaphragm is first cleaned and disinfected, and then the torso model structure is sleeved on the human body. From top to bottom, the head unit 1 is worn, then the chest unit 2, the waist unit 3 and the lower abdomen unit 4 are tied. The electromagnetic output unit 6 is started through the wristwatch control unit, and parameters such as the mode, time, strength and the like are selected on the wristwatch control unit.

### Embodiment 2

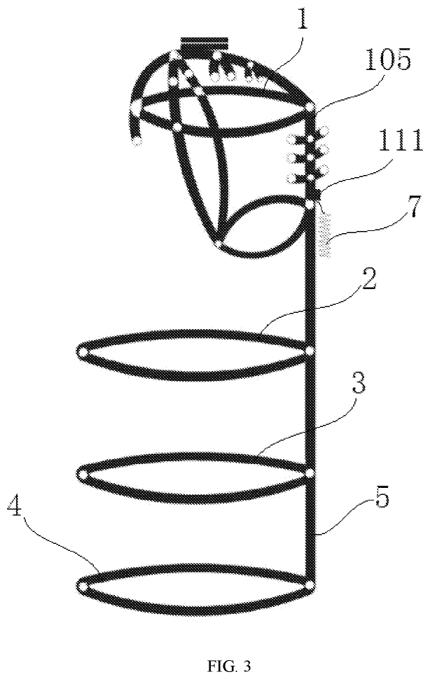

The electromagnetic physiotherapy apparatus of conception and governor vessels, as shown in FIG. 3, is different from Embodiment 1 in that the torso model structure in the present embodiment is an inflatable structure, that is, the head unit 1, the chest unit 2, the waist unit 3, the lower abdomen unit 4 and the main torso 5 are all formed by winding an empty gasbag.

In order to inflate the torso model structure, the electromagnetic physiotherapy apparatus in the present embodiment further includes an inflation device. The main vertical belt 105 is provided with the joint 111, that is, the joint 111 is arranged at the head unit 1, so that the electromagnetic diaphragm can be tightly contact with the surface of the human body by means of the inflation adjustment of the inflation device, which is greatly different from the traditional sticky patch.

In the present embodiment, the electromagnetic diaphragm arranged on the torso model structure is electrically connected to the electromagnetic output unit 6 through a wire, and the wire is arranged in the empty gasbag.

In addition, the torso model structure in the present embodiment further includes the auxiliary lead string 7, and when in use, the auxiliary lead string 7 can be tied to four limbs, which takes into account traditional electrode patch auxiliary leads. Specifically, the auxiliary lead string 7 is arranged on the vertical belt 105.

### Embodiment 3

An electromagnetic physiotherapy method of conception and governor vessels includes: enabling a bionic electromagnetism to synchronously act on a neuron area, a block and a chain to promote connections between synapses of the neuron area, the block and the chain. The neuron area includes the prefrontal lobe, the posterior frontal lobe, the parietal lobe, the temporal lobe and the occipital lobe of the brain. The block includes the cerebellum, the epithalamus and hypothalamus, the pituitary, the epiphysis, the pons and the medulla oblongata. The chain includes a spinal cord and a peripheral nervous system.

The physiotherapy method of the present invention enables the electromagnetism to synchronously act on the neuron area, the block and the chain to cause the neuron area, the block and the chain to produce a synergistic effect, which has the better physiotherapy effect far beyond the traditional point-to-point patch electrode physiotherapy method. The method of the present invention can invigorate health effectively, improve functions of respective tissues and organs, regulate the functions of heart and brain vessels, and comprehensively stimulate neurons to promote rehabilitation of the vegetative patient.

It should be pointed out that, for those having ordinary skill in the art, several modifications and improvements may also be made without departing from the inventive concept of the present invention, and these modifications and improvements shall fall within the scope of protection of the present invention.

What is claimed is:

1. An electromagnetic physiotherapy apparatus of conception and governor vessels, comprising:
an electromagnetic generating device, and
a torso model structure, wherein the torso model structure is electrically connected to the electromagnetic generating device; the torso model structure is configured to be worn by a user, and the torso model structure is provided with electromagnetic diaphragms at positions corresponding to acupuncture points and tissues of a human body.

2. The electromagnetic physiotherapy apparatus of claim 1, wherein the torso model structure comprises a head unit, a chest unit, a waist unit and a lower abdomen unit; the head unit, the chest unit, the waist unit and the lower abdomen unit are connected through a main torso.

3. The electromagnetic physiotherapy apparatus of claim 2, wherein the head unit has a helmet-shaped structure, and the head unit is provided with a plurality of first electromagnetic diaphragms at positions respectively corresponding to a prefrontal lobe, a posterior frontal lobe, a parietal lobe, a temporal lobe, an occipital lobe, a cerebellum, epithalamus and hypothalamus, a pituitary, an epiphysis, a pons and a medulla oblongata of a brain of the human body; and
each of the chest unit, the waist unit and the lower abdomen unit has an annular structure, and the annular structure is provided with at least one second electromagnetic diaphragm.

4. The electromagnetic physiotherapy apparatus of claim 3, wherein the head unit comprises:
an arc belt, wherein the arc belt corresponds to a top of a head of the human body;
a first annular belt, wherein the first annular belt is connected to the arc belt, and the first annular belt is arranged around the head of the human body in one circle;
a second annular belt, wherein the second annular belt is arranged around a neck of the human body in one circle;
a third annular belt, wherein a top of the third annular belt is connected to the arc belt, and a bottom of the third annular belt is connected to the second annular belt; and
a vertical belt, wherein a top of the vertical belt is connected to the first annular belt, and a bottom of the vertical belt is connected to the second annular belt;
wherein each of the arc belt, the first annular belt, the second annular belt, the third annular belt and the vertical belt is provided with the plurality of first electromagnetic diaphragms.

5. The electromagnetic physiotherapy apparatus of claim 4, wherein the arc belt is further connected to a first branch belt and a second branch belt, and the first branch belt and the second branch belt are perpendicular to an extension direction of the arc belt;
the vertical belt is further connected to a third branch belt, a fourth branch belt and a fifth branch belt, and the third branch belt, the fourth branch belt and the fifth branch belt are perpendicular to an extension direction of the vertical belt; and
two ends of the first branch belt, two ends of the second branch belt, two ends of the third branch belt, two ends of the fourth branch belt and two ends of the fifth branch belt are separately provided with a first electromagnetic diaphragm of the plurality of first electromagnetic diaphragms.

6. The electromagnetic physiotherapy apparatus of claim 2, wherein the torso model structure further comprises an auxiliary lead string, wherein the auxiliary lead string is configured to be tied to four limbs.

7. The electromagnetic physiotherapy apparatus of claim 2, wherein the torso model structure is an inflatable structure, and the torso model structure is inflated to tightly press the electromagnetic diaphragms against the human body; the torso model structure is communicated with an inflation device; the torso model structure is provided with a joint, and the inflation device is communicated with the joint.

8. The electromagnetic physiotherapy apparatus of claim 2, wherein the electromagnetic generating device comprises:
an electromagnetic output unit, wherein the electromagnetic output unit is electrically connected to the torso model structure; and
a power source, wherein the power source is electrically connected to the electromagnetic output unit.

9. The electromagnetic physiotherapy apparatus of claim 1, further comprising:
a control unit, wherein the control unit communicates with the electromagnetic generating device to input physiotherapy modes, time and strength signals to control the electromagnetic generating device.

10. An electromagnetic physiotherapy method of conception and governor vessels, comprising: enabling a bionic electromagnetism to synchronously act on a neuron area, a block and a chain to promote connections between synapses of the neuron area, the block and the chain;
wherein the neuron area comprises a prefrontal lobe, a posterior frontal lobe, a parietal lobe, a temporal lobe and an occipital lobe of a brain; the block comprises a cerebellum, epithalamus and hypothalamus, a pituitary, an epiphysis, a pons and a medulla oblongata; and the chain comprises a spinal cord and a peripheral nervous system.
